(19) United States
(12) Patent Application Publication
(10) Pub. No.: US 2010/0279429 A1
(43) Pub. Date: Nov. 4, 2010

(54) Method and Apparatus for Increasing the Sensitivity of a Biosensor Used in a Planar Waveguide

(75) Inventors: Karlheinz Hildenbrand, Krefeld (DE); Alexandre Izmailov, Toronto (CA); Stephan Schwers, Koln (DE)

Correspondence Address: Hildebrand, Christa;Norris McLaughlin & Marcus PA, 875 Third Avenue, 8th Floor, New York, NY, 10022, US

(73) Assignee: BAYER TECHNOLOGY SERVICES GMBH, Leverkusen (DE)

(21) Appl. No.: 12/741,653

(22) PCT Filed: Nov. 6, 2008
(86) PCT No.: PCT/US08/82544

§ 371 Date: Jul. 6, 2010

Related U.S. Application Data
(60) Provisional application No. 60/986,037, filed on Nov. 7, 2007.

(51) Int. Cl. G01N 1/00; G01N 33/48

(52) U.S. Cl. 436/174; 422/68.1

(57) ABSTRACT

Systems, methods and apparatus are provided for mixing an analyte in a planar waveguide cartridge. The invention includes adding magnetic particles to an analyte containing one or more types of target molecules; inserting the analyte and magnetic particles into the cartridge; and moving a magnetic filed proximate to and around the cartridge containing the analyte and magnetic particles, wherein the movement of the magnet field causes movement in the analyte.

### FIELD OF THE INVENTION

The present invention relates to planar waveguide technology, and more specifically to methods and apparatus for increasing the sensitivity of a biosensor used in a planar waveguide.

### BACKGROUND

Biosensors are devices used to detect desired biological molecules. Biosensors typically function by combining a biological component with a physiochemical detector component. A biosensor may include three parts: the biological material to be sampled, a detector element (e.g., may include a physiochemical reaction mechanism) and a transducer, for associating the biological material with the detector element. A simple example of a biosensor is the canary in a cage brought into a coal mine, used by miners to warn of gas. Blood sugar monitors used by diabetics include a biosensor for the detection of blood glucose concentration. Other examples of biosensors include but are not limited to sensors for detecting other health related targets, environmental applications (e.g., sensors for the detection of pesticides and river water contaminants), remote sensing of airborne bacteria (e.g., in counter-bioterrorist activities), detection of pathogens, determining levels of toxic substances before and after bioremediation, and detection and determination of organophosphate.

A waveguide is a structure for guiding radiation (e.g., light, etc.) and may enable the excitation of molecules attached to the surface of the waveguide or in its very close proximity by an evanescent field originated by guided radiation. A planar waveguide guides a plane of radiation of limited width in one direction. Planar waveguide (hereinafter “PWG”) sensors may be used with biosensors to detect target biological matter. Conventionally, the PWG sensor is brought in contact with a sample (analyte) containing biological molecules of interest. The biological molecules of interest (hereinafter “target molecules”) may bind to the capture probes on the PWG sensor during a hybridization process. The single PWG sensor may have multiple types of capture probes for attracting more than one type of target molecule in the hybridization process. The PWG sensor may be housed within a cartridge having a cover. A narrow space between the upper surface of the PWG sensor and the cartridge cover is filled with analyte. The space allows the target molecules in the analyte to contact and hence hybridize to the PWG sensor. The conventional hybridization process may require an extended period of time. Thus, what is needed are systems and methods for accelerating the process and reducing the hybridization time.

### SUMMARY OF THE INVENTION

In some aspects of the present invention, an apparatus is provided for increasing the sensitivity of a biosensor having a planar waveguide cartridge having a cover and adapted to house a planar waveguide sensor, an analyte sample disposed between the planar waveguide sensor and the cover, wherein the analyte includes one or more magnetic particles, and a magnetic field adapted to move the one or more magnetic particles within the analyte.

In other aspects of the present invention a method for mixing an analyte in a planar waveguide cartridge is provided. The method comprises adding one or more magnetic particles to an analyte containing one or more types of target molecules, introducing the analyte and magnetic particles to the cartridge, applying an electro-magnetic field proximate the cartridge containing the analyte and magnetic particles and removing the electro-magnetic field proximate the cartridge containing the analyte and magnetic particles, wherein the application and removal of the electro-magnetic field causes movement in the analyte.

In yet other aspects of the present invention a system is used in diagnostic screening. The system comprises a planar waveguide cartridge having a cover and adapted to house a planar waveguide probe, an analyte sample disposed between the planar waveguide probe and the cover, wherein the analyte includes one or more magnetic particles, a magnetic field adapted to move the one or more magnetic particles within the analyte and a sensor adapted to determine the presence of a predetermined amount target molecules.

Other features and aspects of the present invention will become more fully apparent from the following detailed description, the appended claims, and the accompanying drawings.

### BRIEF DESCRIPTION OF THE DRAWINGS

FIG. 1 is a perspective view of a cartridge containing a PWG sensor and analyte in accordance with embodiments of the present invention.

FIG. 2 is a perspective view of an embodiment of a PWG sensor with analyte in accordance with embodiments of the present invention.

FIG. 3 is a perspective view of a PWG sensor with analyte and the mixing apparatus in accordance with embodiments of the present invention.

FIG. 4 is a flowchart illustrating an exemplary method for mixing an analyte in a planar waveguide cartridge in accordance with embodiments of the present invention.

FIG. 5 is a flowchart illustrating an exemplary method for mixing an analyte in a planar waveguide cartridge in accordance with embodiments of the present invention.

### DETAILED DESCRIPTION

The inventors of the present invention have determined that a problem that exists with conventional PWG technology is that not all of the analyte containing target molecules contacts the capture probes in a PWG sensor. The exchange of molecules between the different parts of the analyte volume is very slow. One reason for this may be that the space between the upper surface of the PWG sensor and the cover of the cartridge containing the analyte is very narrow, therefore movement of the target molecules in the analyte may be restricted. Thus, it may take a long time for sufficient numbers of target molecules to come into contact with any given capture probe on the PWG surface, and consequently only a small fraction of target molecules can hybridize to the PWG sensor. Alternatively or additionally, the volume of analyte in the cartridge may still contain a considerable amount of molecules of interest that do not get hybridized to the PWG sensor, thereby limiting the sensitivity of the PWG sensor. Among other things, the present invention addresses this problem in particular.

The present invention provides systems, apparatus and methods for increasing the sensitivity of a planar waveguide (PWG) sensor and/or reducing the time required to hybridize molecules of interest to the capture probes. In particular, the improved PWG sensor of the present invention may be used, for example, in cancer diagnostics to more reliably test for the presence of a plurality of different genes, for example, HER-2/neu, estrogene receptor, progesterone receptor, MYC, p53, RAF, TRK, BRCA1 or BRCA2. The invention provides for the increased sensitivity and/or reduced hybridization time by gently stirring the analyte to increase contact between the PWG sensor and the target molecules in the entire volume of liquid without disturbing the molecules of interest that have already hybridized. In particular, the inventors have determined that the small scale of the PWG technology is best served by a special means to create controlled movement within the analyte. Additionally, the movement of analyte in the cartridge is preferably a gentle movement to prevent the destruction of the capture probes on the surface of the PWG sensor, as well as to minimize the removal of already hybridized target molecules from the PWG sensor. In the present invention, magnetic or magnetically susceptible (i.e., paramagnetic) particles may be added to the analyte before the analyte is inserted in the cartridge. (Note that throughout this specification and the appended claims, the term “magnetic particles” is used to refer to both permanent magnetic particles and paramagnetic particles, unless otherwise stated.) A magnetic field may be introduced to move the magnetic particles within the analyte as the cartridge is held stationary. As the magnetic particles move within the analyte, other molecules within the analyte may be displaced and caused to move. The movement of the other molecules may increase the rate of delivery of the target molecules to the capture probes on the PWG sensor and hence increase the rate of hybridizing to the capture probes on the PWG sensor. In other words, if the analyte were not stirred, the concentration of target molecules in the layer of analyte closest in proximity to the capture probes of the PWG sensor would be exhausted/reduced due to the fact that some of these molecules would be hybridizing to the surface of the PWG sensor. Mixing the analyte allows the liquid to re-homogenize and re-fill the layer of analyte in closest proximity to the PWG sensor with target molecules, thus compensating for the target molecules being removed from the analyte through hybridization to the PWG sensor. As a result, the concentration of the target molecules may be effectively increased in the layer of analyte in closest proximity to the PWG sensor, which leads to an increased rate of delivery of these target molecules to the capture probes of the PWG sensor surface, and as a further result leads to an increase in the sensitivity of the biosensor. Additionally, it is noted that hybridized bonds between the target molecules and the PWG sensor probes may not necessarily be strong enough to keep the target molecules hybridized to the PWG sensor during agitation of the analyte by other means (e.g., shaking or other motion of the cartridge), which is not the case when moving magnetic particles are used.

Conventionally, magnetic particles have been used for capturing biological molecules on their surface with the intent of separating components of a solution. However, in the present invention, these magnetic particles may be coated with a material to prevent the magnetic particles from binding with the target molecules. If the binding were to take place, the PWG sensor and hybridization process would be competing with the magnetic particles for the target molecules, decreasing the speed and sensitivity of the PWG sensor. Additionally, the size and concentration of the magnetic particles may be chosen to provide a pre-set amount of movement of the analyte layers. Further, the magnetic field shape and strength may also be selected and adjusted to achieve a particular result (e.g., a degree of analyte mixing or motion). For example, in some embodiments, the rate of change of the magnetic field may be slow enough to allow larger displacement of the paramagnetic or magnetic particles and allow the molecules in the analyte to flow within the cartridge. Another feature of the magnetic particles may be that the magnetic particles may not remove molecules of interest from the capture probes of the PWG sensor which have already hybridized to the capture probes of the PWG sensor.

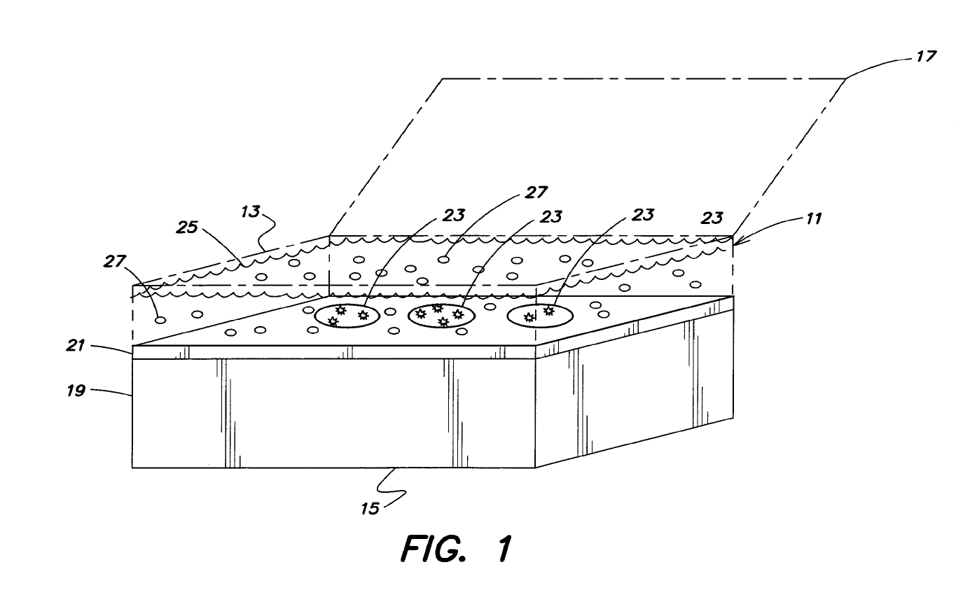

Turning to FIG. 1, a perspective view of a planar waveguide cartridge 11 used with planar waveguide technology is depicted. The cartridge 11 may be embodied as a box-like structure suitable to contain analyte. Herein, the cartridge 11 is depicted as a cube-shaped enclosure. However, this is for purposes of example only, and the cartridge 11 may be formed in any other practicable shape. The cartridge 11 may include a top 13 and a bottom 15, on opposing sides thereof. A cover 17, shown herein in an open position, may cover the top 13 of the cartridge 11 when the cartridge 11 is in the closed position. The cover 17 may be used to selectively keep the contents of the cartridge 11 intact. A substrate 19 may be positioned in the bottom 15 of the cartridge 11. The substrate may be made of glass, for example, or any other suitable material able to transmit the radiation used with PWG technology. A PWG sensor 21 or waveguiding layer may be positioned on top of the substrate 19. The PWG sensor 21 may include at least one capture probe 23 on the surface opposite the surface contacting the substrate 19. An analyte 25, or liquid sample, including one or more target molecules 27 may be placed in the cartridge 11 on top of the PWG sensor 21, such that a portion of the analyte 25 may be in contact with the capture probes 23. The target molecules 27 may, for example, be a DNA or an RNA fragment. Other types of target molecules may be provided. A plurality of different types of analyte 25 may be used, for example, proteins, DNA or RNA extracted from blood, serum, plasma, tissue, sputum, buccal swabs or feces.

In the example depicted herein, there are three capture probes 23. However, this is for purposes of example only, and a much larger plurality of capture probes 23 commensurate with PWG technology may be used. The capture probes 23 may be used to attract and bind to the target molecules 27 in the analyte 25 upon application of the radiation through the waveguide 19. Herein, only one type of target molecule 27 is shown. However, this is for purposes of example only, and a single PWG sensor 21 may be used to attract a plurality of different types of target molecules 27. The target molecules 27 may hybridize or attach themselves to the capture probes 23. The radiation used with PWG technology may excite the label (dye molecule, for example) attached to target molecules 27. The greater the number of target molecules 27 that can be made to hybridize to the capture probes 23, the greater the signal produced by the biosensor. By increasing in the sensitivity of the biosensor, the PWG sensor 21 becomes more effective and accurate in making diagnostic determinations.

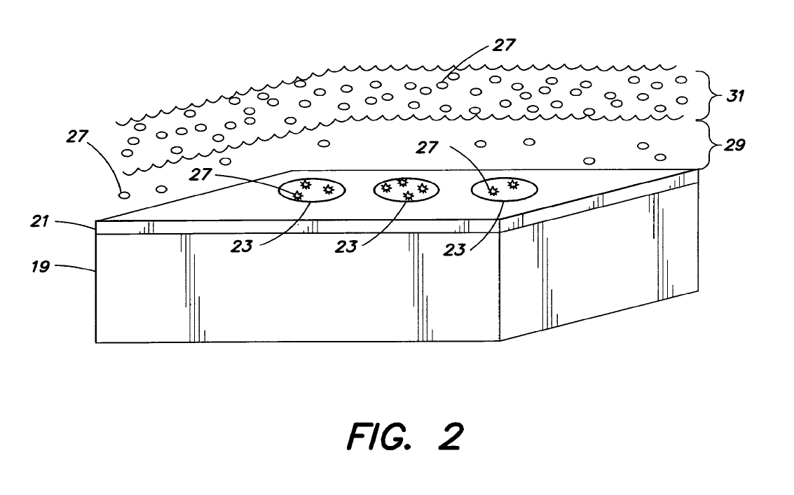

FIG. 2 is a perspective view of the inside of the cartridge 11. The sidewalls have been omitted to more clearly illustrate the invention. When the cover 17 of the cartridge 11 is in the closed position (not shown in FIG. 2), there may be a limited amount of space between the PWG sensor 21 and the cover 17. The space may be approximately 0.05 mm to approximately 0.2 mm. Other amounts of space may be provided. The analyte 25 may be placed in this limited space and because the space is limited, the target molecules 27 within the analyte 25 filling the space may be restricted in their movement within the analyte 25. For the sake of description, the analyte 25 has a first layer 29 in closest proximity to the PWG sensor 21 and a second layer 31 sandwiched between the first layer 29 and the cover 17. However, the analyte 25 does not necessarily have well-defined layers.

Before hybridization begins, the target molecules 27 may be homogenously distributed throughout the analyte layers 29, 31. As depicted in FIG. 2 however, once the hybridizing process begins, many of the target molecules 27 in the first layer 29 bind the capture probes 23, depleting the first layer 29 of target molecules 27. The target molecules 27 will naturally move or diffuse from an area of high concentration to an area of low concentration to create an equalization of concentrations. However, because of the restricted movement in the analyte 25, it may take a long time for sufficient numbers of target molecules 27 to move from the second layer 31 to the first layer 29 and therefore come in contact with a capture probe 23. Consequently, only a small fraction of target molecules 27 may hybridize to the capture probes 23 in a given amount of time.

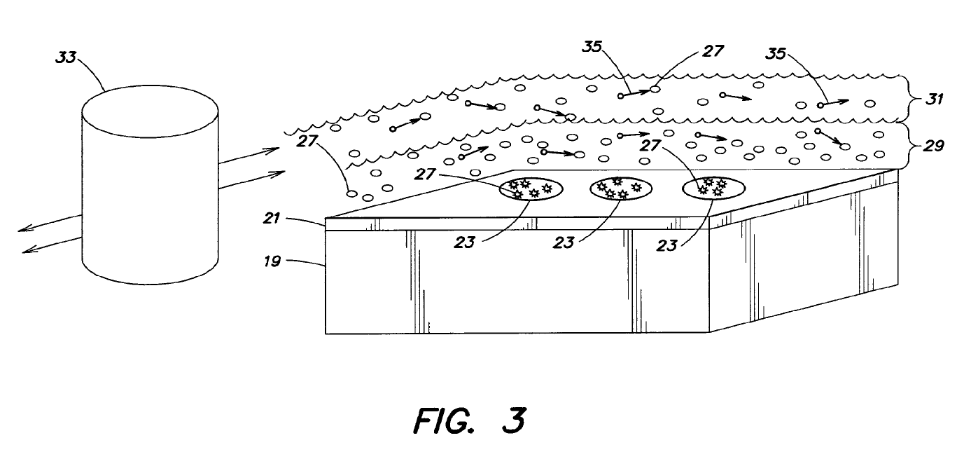

Turning to FIG. 3, a perspective view of an embodiment of the inside of the cartridge 11 of the present invention is depicted, again without sidewalls for clarity. Herein, a plurality of magnetic particles 35 have been added to the analyte 25. A magnet 33 may be located outside of the cartridge 11. The magnet 33 may be moved in a side-to-side motion, as indicated by the directional arrows. As the magnet 33 moves, the magnetic field emanating from the magnet 33 causes the magnetic particles 35 in the analyte 25 to move. As the magnetic particles 35 move, they may displace other molecules in the analyte 25, such as the target molecules 27, for example, and may cause these other molecules to move. The movement of the target molecules 27 in the analyte 25, causes the target molecules in the second layer 31 to move into the first layer 29 to replenish the target molecules 27 that have already hybridized to the probes 23 and equalize the concentration in the first layer 29. As described above with respect to FIG. 2, the concentration of target molecules 27 in the first layer 29 may be depleted due to the hybridization of the target molecules 27 to the capture probes 23. The effective sensitivity of the PWG sensor 21 may thus be increased by replenishing the concentration of target molecules 27 in the first layer 29 with target molecules 27 from the second layer 31, because this may result in an increase in the number of target molecules 27 susceptible to hybridizing to the capture probes 23.

The magnetic particles 35 may vary in size and shape depending on the optimum amount of movement in the analyte 25. The magnetic particles 35 may have a size in the range of approximately 0.05 micrometer to approximately 20 micrometers. In some embodiments, the magnetic particles 35 may include flat or concave surfaces and/or be elongate-shaped to increase the amount of molecules displaced as the magnetic particles 35 move through the analyte 25. In some embodiments, the magnetic particles 35 may be coated to make the magnetic particles 35 inert and non-reactive with the molecules in the analyte 25. The coating may be, for example, a polymer made from anionic polyelectrolytes. Other materials may be used to make the coating. The anionic polyelectrolytes may be, for example, dextranesulfate NA salt and polyacrylic acid NA salt. In addition to being non-reactive, the magnetic particles 35 may also be formed such that they may not mechanically remove hybridized target molecules 27 from the capture probes 23. In some embodiments, a second, smaller magnetic field may be employed to repel (or attract) the magnetic particles 35 away from the capture probes 23 to further prevent the magnetic particles 35 from mechanically removing hybridized target molecules 27.

The movement of the magnetic particles 35 is affected by the magnet 33. The magnet 33 may be close enough in proximity to the magnetic particles 35 to cause the magnetic particles 35 to move. The proximity of the magnet 33 to the magnetic particles 35 may determine the strength of the magnetic field acting on the magnetic particles 35. Additionally, or alternatively, the size of the magnet 33 may also determine the size of the magnetic field acting on the magnetic particles 35.

The magnet 33 may be an electro-magnet (e.g., a solenoid) that may be turned “on” and “off.” The magnetic particles 35 may move in response to the electro-magnet, (and hence the magnetic field,) being turned “on” and “off.” The movement of the magnetic particles 35 may in turn cause movement in the analyte 25 as described above. Alternatively, the magnet 33 may be a permanent magnet with a constant field that moves with the magnet 33. The permanent magnet 33 may be moved in a side-to-side motion (as indicated by directional arrows in FIG. 3) near a non-moving cartridge 11, causing the magnetic particles 35 within the cartridge 11 to move, which in turn causes movement in the analyte 25. Other motions of the magnet are possible to effect the desired motion of the magnetic particles 35. For example, the magnet 33 may be moved around the cartridge 11.

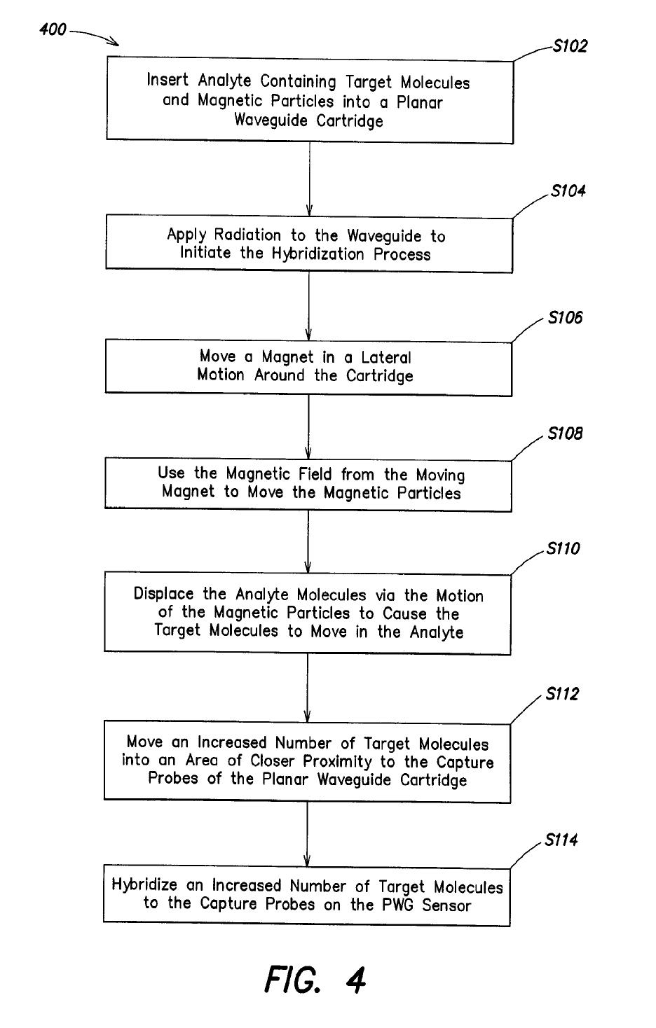

Turning to FIG. 4, a flowchart illustrating an example method 400 of the present invention is depicted. In step S102, analyte containing target molecules and magnetic particles is introduced into a planar waveguide cartridge. In step S104, radiation is applied to the waveguide to initiate the hybridization process. A permanent magnet is moved in a side-to-side motion around the cartridge in step S106. The magnetic field from the moving permanent magnet causes the magnetic particles to move in step S108. In some embodiments, the changing direction of motion of the magnetic field is used to cause the motion of the magnetic particles to change direction. In some embodiments, the magnet may be moved completely around the cartridge which may tend to make the magnetic particles moving in a spiral or circular pattern. In step S110, the moving magnetic particles displace the analyte molecules, including the target molecules, and cause the target molecules to move in the analyte. An increased number of target molecules move into an area of closer proximity to the capture probes in step S112. In step S114, an increased number of target molecules hybridize to the capture probes on the PWG sensor.

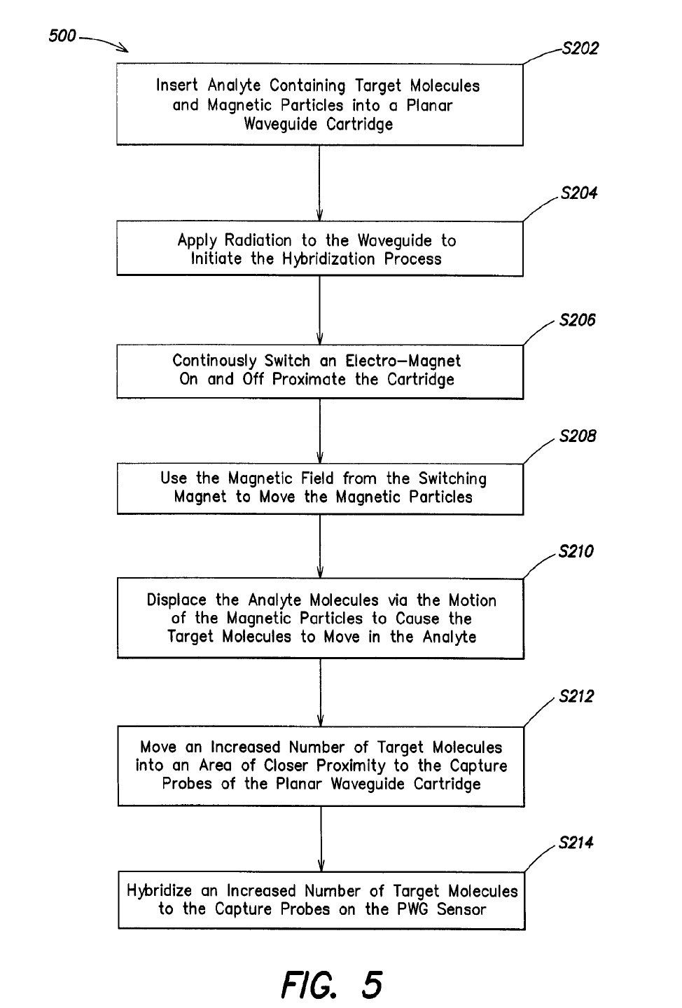

Turning to FIG. 5, a flowchart illustrating a second example method 500 of the present invention is depicted. In step S202, analyte containing target molecules and magnetic particles is introduced into a planar waveguide cartridge. In step S204, radiation is applied to the waveguide to initiate the hybridization process. An electro-magnet proximate the cartridge is continuously switched on and off to create a changing magnetic field in step S206. In some embodiments, the rate of switching of the electro-magnet is in the range of approximately 0.1 Hz to approximately 1 Hz. The strength of the magnetic field may be in the range of approximately 200 gauss to approximately 2000 gauss. In some embodiments, the analyte may include a ferrofluid. The magnetic field from the switching electro-magnet causes the magnetic particles to move in step S208. In some embodiments, the polarity of the magnetic field may be reversed in alternating power cycles to change the direction of motion of the magnetic particles. In some embodiments, the electro-magnet remains on and is moved as described above with respect to the permanent magnet used in method 400. In step S210, the moving magnetic particles displace the analyte molecules, including the target molecules, and cause the target molecules to move in the analyte. An increased number of target molecules move into an area of closer proximity to the capture probes in step S212. In step S214, an increased number of target molecules hybridize to the capture probes on the PWG sensor.

The foregoing description discloses only exemplary embodiments of the invention. Modifications of the above disclosed apparatus and method which fall within the scope of the invention will be readily apparent to those of ordinary skill in the art.

Accordingly, while the present invention has been disclosed in connection with exemplary embodiments thereof, it should be understood that other embodiments may fall within the spirit and scope of the invention, as defined by the following claims.

The invention claimed is:

1. An apparatus for increasing the sensitivity of a biosensor comprising:
a biosensor cartridge including a cover and adapted to house a biosensor;
an analyte chamber within the biosensor cartridge disposed between the biosensor and the cover, wherein the analyte chamber is adapted to receive analyte that includes a plurality of magnetic particles; and
a magnetic field adapted to move the magnetic particles within the analyte.

2. The apparatus of claim 1, wherein the biosensor includes a planar waveguide sensor.

3. The apparatus of claim 2, wherein a surface of the planar waveguide sensor exposed to the analyte includes one or more capture probes.

4. The apparatus of claim 3, wherein the analyte includes one or more types of target molecules adapted to hybridize to the one or more capture probes.

5. The apparatus of claim 1, wherein the magnetic particles are coated with a coating to make the magnetic particles non-reactive with the analyte.

6. The apparatus of claim 5, wherein the coating is made from anionic polyelectrolytes.

7. The apparatus of claim 6, wherein the anionic polyelectrolytes are at least one of dextranesulfate Na salt and polyacrylic acid Na salt.

8. The apparatus of claim 5, wherein the coated magnetic particles do not mechanically remove hybridized target molecules from the one or more capture probes.

9. The apparatus of claim 1, wherein the magnetic particles are a predetermined size.

10. The apparatus of claim 1, wherein the magnetic field is produced by an electro-magnet.

11. The apparatus of claim 10, wherein the magnetic field is adapted to move the magnetic particles by switching the electro-magnet on and off proximate the cartridge.

12. The apparatus of claim 11, wherein the cartridge remains stationary.

13. The apparatus of claim 1, wherein the magnetic field is produced by a magnet.

14. The apparatus of claim 13, wherein the magnetic field is adapted to move the one or more magnetic particles by moving the magnet proximate the cartridge.

15. The apparatus of claim 13, wherein the magnet is a solenoid.

16. The apparatus of claim 14, wherein the cartridge adapted to remain stationary.

17. A method for mixing an analyte in a planar waveguide cartridge comprising:
adding magnetic particles to an analyte containing one or more types of target molecules;
inserting the analyte and magnetic particles into a planar waveguide cartridge;
applying a magnetic field proximate the cartridge containing the analyte and magnetic particles; and
removing the magnetic field from the cartridge containing the analyte and magnetic particles, wherein the application and removal of the magnetic field causes movement in the analyte.

18. The method of claim 17 further comprising:
applying radiation to the planar waveguide cartridge to increase the hybridization of the target molecules to one or more capture probes in the planar waveguide cartridge.

19. A method for mixing an analyte in a planar waveguide cartridge comprising:
adding a plurality of magnetic particles to an analyte containing one or more types of target molecules;
inserting the analyte and magnetic particles into a planar waveguide cartridge; and
moving a magnet having a magnetic field proximate and around the cartridge containing the analyte and magnetic particles, wherein the movement of the magnet causes movement in the analyte.

20. The method of claim 19, wherein the cartridge is held stationary.

21. The method of claim 19, wherein the magnet is moved from side-to-side in a horizontal plane.

22. A system for use in diagnostic screening comprising:
a planar waveguide cartridge having a cover and adapted to house a planar waveguide sensor;
an analyte chamber disposed between the planar waveguide probe and the cover, wherein the analyte chamber is adapted to contain analyte that includes a plurality of magnetic particles;
a magnetic field adapted to move the magnetic particles within the analyte; and
a detector adapted to determine the presence of a predetermined amount target molecules.

23. The system of claim 22, wherein the surface of the planar waveguide sensor exposed to the analyte includes one or more capture probes.

24. The system of claim 23, wherein the analyte includes one or more types of target molecules adapted to hybridize to the one or more capture probes.

25. The system of claim 22, wherein the one or more magnetic particles are coated with a coating to make the one or more magnetic particles inert.

26. The system of claim 22, wherein the magnetic field is supplied by an electro-magnet.

27. The system of claim 26, wherein the magnetic field is adapted to move the magnetic particles by switching the electro-magnet on and off proximate the cartridge.

28. The system of claim 22, wherein the magnetic field is produced by a magnet.

29. The system of claim 28, wherein the magnetic field is adapted to move the magnetic particles by moving the magnet proximate the cartridge.
